(19) United States
(12) Patent Application Publication
(10) Pub. No.: US 2023/0317255 A1
(43) Pub. Date: Oct. 5, 2023

(54) DYNAMIC ADMINISTRATIVE SYSTEM FOR HEALTHCARE AND TELEHEALTH MANAGEMENT

(71) Applicant: Murlynn Crystal Lee, Pheonix, AZ (US)

(72) Inventor: Murlynn Crystal Lee, Pheonix, AZ (US)

(21) Appl. No.: 17/707,892

(22) Filed: Mar. 29, 2022

(51) Int. Cl. G16H 40/20; G06Q 10/10; H04L 9/40; H04L 67/12; H04L 67/52

(52) U.S. Cl. CPC G16H 40/20; G06Q 10/1095; H04L 63/0428; H04L 63/166; H04L 67/12; H04L 67/52

(57) ABSTRACT

The present disclosure describes a system for integrating dynamic scheduling and management of appointments and telehealth conferences. The system has at least one client device associated with a user, wherein the at least one client device comprises a graphical user interface (GUI) that allows a user to input a request and a plurality of user variables, an encryption server in communication with the management server, wherein the encryption server is configured to encrypt and decrypt the user inputs, and a queue server in communication with the management server and encryption server, wherein the queue server is configured to receive the plurality of variables, wherein the queue server comprises a global positioning (GPS) module configured to ascertain a location of the user and the physician, and wherein using the location and at least one of the plurality of variables, the queue server is configured to automatically determine whether the appointment is an in-person appointment or a telehealth appointment and notify both the user and the physician.

### FIELD OF THE INVENTION

The present invention generally relates to a system and method for administrating, scheduling and providing healthcare services in areas with little or no access to healthcare facilities. More specifically, the present invention relates to a system and method for secure connection and routing of data for healthcare service industry (e.g., doctors/physicians) that allows scheduling and management of appointments and telehealth conferences for patients and doctors with little or no access to healthcare facilities.

### BACKGROUND

By any measure, health care for Native Americans lags behind other groups, despite a legal obligation on the part of the United States to provide health care to Native Americans and Alaska Natives. Native American communities face significant inequity in health care and health status compared to other U.S. populations. Health outcomes for Native Americans are adversely impacted by wholly inadequate access to comprehensive health services. Indeed, Native Americans and Alaska Natives born today have a life expectancy that is approximately 4.4 years less than all races populations in the United States, and they continue to die at higher rates than other Americans in many categories of preventable illness, including chronic liver disease and diabetes, and chronic lower respiratory diseases.

The Indian Health Service (IHS)—an agency within the U.S. Department of Health and Human Services—provides care to over 2.2 million Native Americans across the country. Although IHS fulfills treaty responsibilities to provide health care for members of more than 560 recognized tribes, Congress has consistently underfunded the agency, forcing hospital administrators to limit the services offered. As a result, tribal members have a different health care reality than many other U.S. citizens.

Against this background, there are overarching challenges in health care that further exacerbate access to care for Native Americans. For example, a common challenge in many rural communities is the shortage of medical personnel—a problem that is even more severe in tribal communities, especially those in remote reservation locations.

The inability to see a physician in time pose serious health risks for patients as it could mean the difference between catching a disease early on or too late. In addition, an inefficient scheduling process can wreak havoc and raise stress levels for both a health systems' staff and patients, particularly in Native communities.

Furthermore, Native communities may be in remote locations without access to reliable networked coverage and Wi-Fi. Just over half of Native Americans living Oil reservations or other tribal lands with a computer have access to high-speed Internet service, according to new estimates from the U.S. Census Bureau.

The low rate of subscription to a high-speed Internet service—53 percent—in these often rugged, rural areas underscores the depth of the digital divide between Native American country and the rest of the U.S. Between 2013 and 2017, 82 percent a households nationally with a computer reported having a subscription to a broadband Internet service.

The latest data from the bureau's American Community Survey also show a stark national gap in high-speed Internet subscription rates between Native Americans generally (67 percent) and those who do not identify as Native American or Alaska Native (82 percent).

Therefore, there is a need for a system and method that provides an integrated service combining both traveling physician scheduling and telehealth to enable a user to schedule and allow a system manage appointments with the physicians.

### SUMMARY OF THE INVENTION

The following summary of the invention is provided in order to provide a basic understanding of some aspects and features of the invention. This summary is not an extensive overview of the invention and as such it is not intended to particularly identify key or critical elements of the invention or to delineate the scope of the invention. Its sole purpose is to present some concepts of the invention in a simplified form as a prelude to the more detailed description that is presented below.

To achieve the foregoing and other aspects and in accordance with the purpose of the invention, a system and method that integrates dynamic scheduling and management of appointments and telehealth and decision-making modules that provide an output for an optimized scenario based on a plurality of inputs and variables.

Accordingly, this platform provides a system and method that provides integrated services combining telehealth and appointment scheduling with a rolling doctor list based on global positioning of both the patient and physician, network connectivity, a rating system and specialist sorting module among other variables to ensure the patient is seeing the correct or preferred physician.

Further, the platforms provide a HIPPA compliant networked system that protects patient data in areas that have disparate healthcare services and access to physicians.

Further, the platform provides a system and method that enables the patient to schedule and manage appointments with the physicians.

Further, the platform provides a system and method that enables the patient to schedule and manage appointments with the physicians in areas that lack networked coverage.

The system comprises one or more processors, dedicated servers, a memory, and specialized administer module and communications protocol via the dedicated servers in communication with processors, user smart devices and third-party providers. The memory stores program instructions executable by the processors to implement an integrated service. The system enables a patient to schedule an appointment with physicians that is out of service, but is able to notify the patient when in service if telehealth or a physical visit may be provided. Further, the system is configured to make dynamic real-times decisions whether a telehealth conference or an appointment in person should occur.

In one embodiment, the system comprises at least one client device associated with a user, wherein the at least one client device that allows a user to input a request to a physician; a network for receiving the input request to the physician; an client side server in communication with the network, wherein the integrated client side server receives the input request from the physician, and outputs a request to a provider side server to request a physician; a variable sorting module in communication with the network, a queue server in communization with network and configured to queue inputs and monitor a global positing system (GPS) of both a patient and a doctor to ascertain when they are in and out of network service, and plurality of databases comprising a patient database, a physician database, and a queue database all connected to the network; a scheduling module residing on the server, wherein the scheduling module arranges, based on the plurality of variables, an appointment or a telehealth consultation based on said variables.

In an embodiment, a system for integrating dynamic scheduling and management of appointments and telehealth conferences is provided. The system comprises at least one client device associated with a user, wherein the at least one client device comprises a graphical user interface (GUI) that allows a user to input a request and a plurality of user variables, a network for receiving the input request to the physician, an management server in communication with the network, wherein the management server receives the input request from the at least one client device, and outputs a request to a provider-side server, wherein the provider-side server is communication with at least one physician device associated with a physician, wherein the at least one physician device comprises a graphical user interface (GUI) that allows a physician to receive the request at a physician device, an encryption server in communication with the management server, wherein the encryption server is configured to encrypt and decrypt the user inputs; and a queue server in communication with the management server and encryption server, wherein the queue server is configured to receive the plurality of variables, wherein the queue server comprises a global positioning (GPS) module configured to ascertain a location of a user and a physician, wherein using the location and at least one of the plurality of variables, automatically determine whether the appointment is an in-person appointment or a telehealth appointment and notify both the user and the physician.

In another embodiment, a non-transitory computer-readable medium for storing instructions that, when executed on one or more processors, cause the one or more processors to A non-transitory computer-readable medium for storing instructions that, in integrating dynamic scheduling and management of appointments and telehealth conferences, when executed on one or more processors, cause the one or more processors to receive an input from at least one client device associated with a user, wherein the at least one client device comprises a graphical user interface (GUI) that allows the user to input a request and a plurality of user variables; output, via a network a request to a provider-side server, wherein the provider-side server is communication with at least one physician device associated with a physician, wherein the at least one physician device comprises a graphical user interface (GUI) that allows a physician to receive the request at a physician device; encrypt, via an encryption server in communication with the management server, the user inputs; receive the plurality of variables at a queue server in communication with the management server and encryption server; ascertain a location of a user and a physician; use the location and at least one of the plurality of variables, automatically determine whether the appointment is an in-person appointment or a telehealth appointment and notify both the user and the physician.

In another embodiment, a method for integrating dynamic scheduling and management of appointments and telehealth conferences, incorporated in a system including a client device, and a server in communication with the client device, wherein the server comprises a memory to store instructions and a processor coupled with the memory to process the stored instructions, the method comprising the steps of receiving an input from at least one client device associated with a user, wherein the at least one client device comprises a graphical user interface (GUI) that allows the user to input a request and a plurality of user variables; outputting, via a network a request to a provider-side server, wherein the provider-side server is communication with at least one physician device associated with a physician, wherein the at least one physician device comprises a graphical user interface (GUI) that allows a physician to receive the request at a physician device; encrypting, via an encryption server in communication with the management server, the user inputs; receiving the plurality of variables at a queue server in communication with the management server and encryption server; ascertaining a location of a user and a physician; using the location and at least one of the plurality of variables, automatically determine whether the appointment is an in-person appointment or a telehealth appointment and notify both the user and the physician.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF DRAWINGS

The foregoing summary, as well as the following detailed description of the invention, is better understood when read in conjunction with the appended drawings. For the purpose of illustrating the invention, exemplary constructions of the invention are shown in the drawings. However, the invention is not limited to the specific methods and structures disclosed herein. The description of a method step or a structure referenced by a numeral in a drawing is applicable to the description of that method step or structure shown by that same numeral in any subsequent drawing herein.

FIG. 1 illustrates a platform schematic diagram of a system for scheduling and managing healthcare and telehealth services according to an embodiment of the present invention.

FIG. 2 illustrates another schematic diagram for scheduling and managing healthcare and telehealth services according to an embodiment of the present invention.

FIG. 3 shows a combined system and method diagram for scheduling and managing healthcare and telehealth services according to an embodiment of the present invention.

FIG. 4 shows an exemplary schematic diagram for security of the system for scheduling and managing healthcare and telehealth services, according to an embodiment of the present invention.

FIG. 5 shows an exemplary back-end system architecture for scheduling and managing healthcare and telehealth services, according to an embodiment of the present invention.

FIG. 6 shows a step-wise method diagram for scheduling and managing healthcare and telehealth services according to an embodiment of the present invention.

FIG. 7 shows systematic diagram of a variable server according to an embodiment of the present invention.

FIG. 8a shows a step-wise method diagram for scheduling and managing healthcare and telehealth services, according to an embodiment of the present invention.

FIG. 8b shows a continuation of the step-wise method diagram for scheduling and managing healthcare and telehealth services, according to an embodiment of the present invention.

FIG. 8c shows a continuation of the step-wise method diagram for scheduling and managing healthcare and telehealth services, according to an embodiment of the present invention.

FIG. 9 shows another systematic diagram of a variable server according to an embodiment of the present invention.

Other features, advantages, and aspects of the present invention will become more apparent and be more readily understood from the following detailed description, which should be read in conjunction with the accompanying drawings.

### DETAILED DESCRIPTION OF THE INVENTION

The present invention is best understood by reference to the detailed figures and description set forth herein.

It is expected that the present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

Reference will now be made in detail to various embodiments. Each example is provided by way of explanation and is not meant as a limitation and does not constitute a definition of all possible embodiments. The described embodiments are to be considered in all respects only as illustrative and not restrictive. For purposes of illustrating features of the embodiments, a simple example will now be introduced and referenced throughout the disclosure. Those skilled in the art will recognize that this example is illustrative and not limiting and is provided purely for explanatory purposes. An example of a computing system environment is disclosed. The computing system environment is not intended to suggest any limitation as to the scope of use or functionality of the system and method described herein. Neither should the computing environment be interpreted as having any dependency or requirement relating to any one or combination of components illustrated in the exemplary operating environment.

Embodiments of the disclosure are operational with numerous other general purposes or special purpose servers and modules and computing system environments or configurations.

The embodiments of the disclosure may be described in the general context of computer-executable instructions, such as program modules, being executed by a processor or computer. Generally, program modules include routines, programs, objects, components, data structures, etc. that perform particular tasks or implement particular abstract data types. The systems and methods described herein may also be practiced in specialized distributed computing environments where tasks are performed by remote processing devices that are linked through a communications network. In a specialized distributed computing environment, program modules may be located in both local and remote computer storage media including memory unit or storage devices. Tasks performed by the programs and modules are described below and with the aid of figures. Those skilled in the art can implement the exemplary embodiments as processor executable instructions, which can be written on any form of a computer readable media in a corresponding computing environment according to this disclosure.

Components of computer may include, but are not limited to, a processing unit, a system memory, and a system bus that couple various system components including the system memory to the processing unit. The system bus may be any of several types of bus structures including a memory bus or memory controller, a peripheral bus, and a local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus also known as Mezzanine bus.

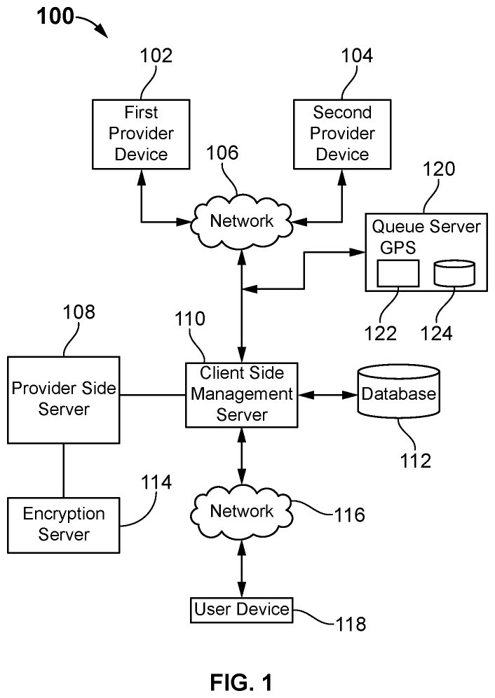

Referring now to FIG. 1, a schematic diagram of a system for scheduling and managing healthcare and telehealth services according to an embodiment of the present invention is shown generally at 100. The system comprises physician device 102 and 104(n+1) associated with a physician and a user or patient device 118 associated with a user. The devices are connected via networks 106 and 116 (or a singular network) and comprises user side management server 110, physician side server 108, encryption server 114, queue server 120 and database 112. The physician device 102 and the user device 118 are in communication with each other via encryption server 114 so that communication is HIPPA compliant and messages can be encrypted and decrypted.

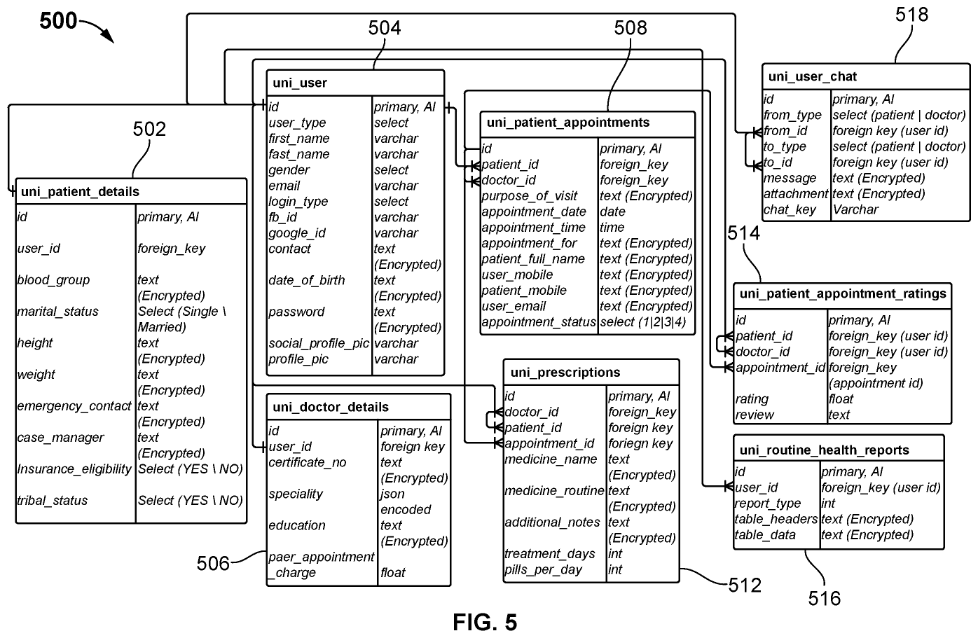

In an embodiment, the physician devices 102 and 104 (n+1) and the user device 118 are smart devices (e.g., smartphones or tablets) running a mobile application but may be a desktop as well. The network 106, 116 may be cellular network, a Wi-Fi network, WiMAX network wireless local area network and the like. The client-side management server 110 is in communication with the physician-side server 108, encryption server 114, queue server 120 and database 112 via the network. The client-side management server comprises a special purposes server and data routing schemes as shown in FIG. 5. The client-side management server 110 may operate via a mainframe, a server farm, and so forth.

In one embodiment, the physician provides a service that is requested via provider-side server 108 based on an input from the user device and client-side server 110. In operation, the servers 108 and 110 enable the user to schedule an appointment, either in person or via telehealth, with a physician. Both servers together (i.e., cloud-based applications) enable the patient and the physician to track a status of the appointment of the user, change appointments unilaterally, and using the queue server 120, the system may automatically decide and send a notification on whether the appointment is in person or via telehealth (discussed in more detail below).

The database 112 comprises a memory to store and organize certain data and variables for use by the servers 110, 108, 112 and 120. In some embodiments, the database stores a plurality of user related information, one or more physician related information and a variety of variables associated with the patient and the physician. The variables may comprise patient location or last known location, past conditions, current conditions, history, and characteristics. The variables, on the physician side, may comprise specialties, ratings, location and last known location, and history among others. Further, variables may comprise preventative care that the system tracks as a self-monitoring aspect of the platform. For example, the patient may input blood sugar/glucose including date measured, blood sugar fasting, blood sugar 1 hour, blood sugar 2 hours, food taken before testing, etc. Other self-monitoring aspects may comprise blood pressure logs including date, time, SBP, DBP, BPM, and notes. Further, weight loss data may include date, weight, chest, waist, and hip measurements. The system may track days sober, food/calorie intake data, sleep data, a d mood tracking,

The encryption server 114 is in communication with the other servers via the network and comprises transport layer security (TLS), a cryptographic protocol configured to provide communications security over a computer network, for example. The TLS protocol provides privacy and data integrity between two or more communicating computer applications and runs in the application layer of the network itself and may be composed of two layers: the TLS record and the TLS handshake protocols, as an example. The encryption server operates in HIPPA compliant fashion so that the physician and the patient can safely share medical information whether it be during a telehealth conference or written information sent to the physician prior to a visit (medical records transfer).

With reference still to FIG. 1, The queue server 120 comprises GPS module 122 and database 124. While a database 124 is shown in embodiments, non-persistent memory may be employed herein. The queue server 120 is configured to store all inputs and outputs from the client-side management server 110 the provider side server 108. The GPS module 122 is configured to communicate with user device 118 and physician device 102 to ascertain location information of the user and the physician. The GPS module 122 may assist in scheduling appointments or deciding if a telehealth appointment is necessary based on the location or past known location of the patient and the physician. In operation, when the patient or the user is out of network or out of cellular service the database 124 is configured to store information and inputs and/or requests on behalf of the patient or the physician so that when they are back in network or back in cellular service, they immediately receive the encrypted message regarding appointments or telehealth appointments. The queue server 120 is further configured to track user location and store a last known location of a user so that when the user or doctor is out of network, the client-side server 108 in the provider-side server 110 may poll the database for location information to ascertain where the user or physician likely is based on numerous previous known locations or appointment locations, home address and other variables. The queue server 120 is configured to, based on the plurality of inputs and variables, make an automated decision as to whether the patient should see the doctor in person or whether it should queue and immediate telehealth conference with a physician at each of the user deice 118 and physician devices 102.

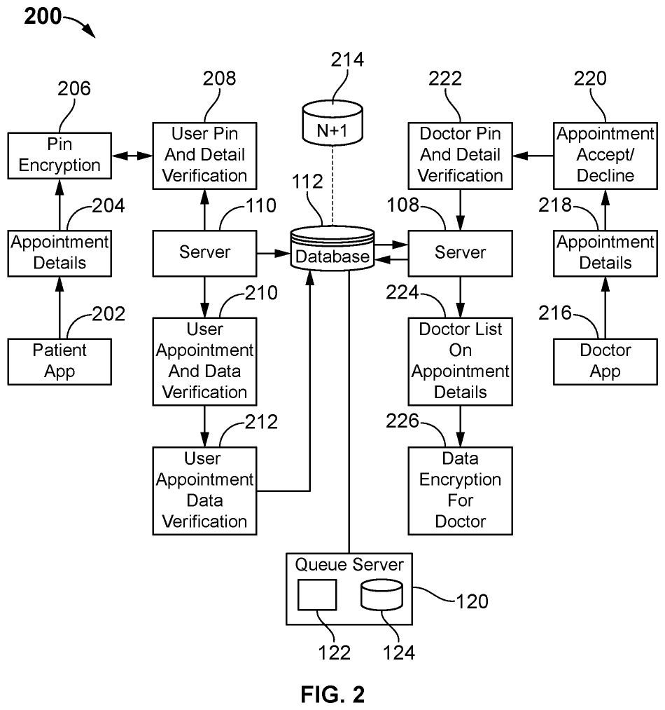

With reference now to FIG. 2, a schematic diagram for scheduling and managing healthcare and telehealth services according to an embodiment of the present invention is provided at 200. The diagram shows both the patient side and physician side. On the patient side, in operation, a patient can open patient application 202 on their smart device. Appointment details module 204 allows the user and the physician to schedule an appointment or a telehealth conference. PIN encryption module 206 (utilizing the encryption server 114) is configured to communicate with multiple databases and encrypt patient information in a HIPPA compliant manner. User PIN and detail verification module, once the patient is logged in, communicates with the queue server 120 and the GPS module 122 and the database 124 or 112 to input user location or last known location of the user/patient. The server 110 is in communication with database 112 and is configured to set user appointment verification module 210. A second encryption module 212 is configured to encrypt the appointment data whether it be an in person visit or telehealth conference.

In operation, a patient can open physician application 216 on their smart device. Appointment details module 218 allow the physician to schedule an appointment or a telehealth conference. An appointment accept/decline module 220 allows the physician to accept a request from the patient, and set an appointment of telehealth conference. Doctor PIN encryption module 122 (utilizing the encryption server 114) is configured to communicate with multiple databases and encrypt patient information in a HIPPA compliant manner via server 114. User PIN and detail verification module, once the doctor is logged in, communicates with the queue server 120, the GPS module 122 and the database 124 or 112 to locate physicians. Physician location may also include or comprise last known location of the physician. A doctor list on appointment details module 204 is in communication with their encryption module 206 to encrypt the appointment list and any patient data that requires encryption.

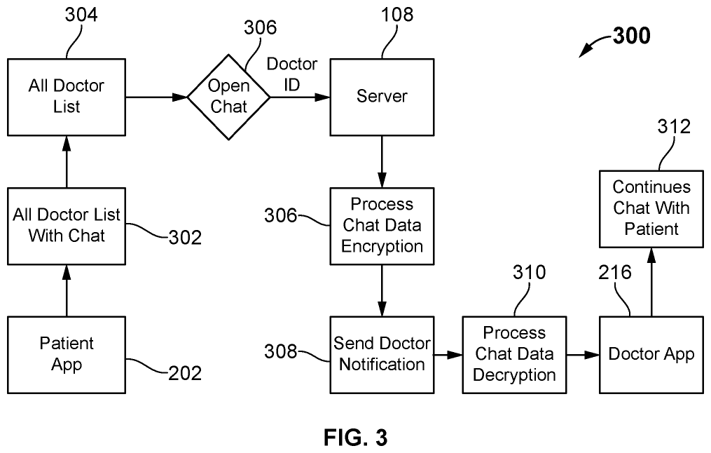

Referring now FIG. 3, a combined system and method diagram for scheduling and managing healthcare and telehealth services according in a chat function is shown generally at 300. In operation a user can open the patient app 202. The system automatically populates a doctor list with chat 302 or doctor list 304 allowing the patient to chat with the doctor either via chat module 306 or automatically ring the doctor via video conference. Once the chat is opened 306, the user and doctor may chat via server 108 once the doctor is identified by the module. A process chat data encryption 306 may be further employed via encryption server 114. Once encrypted a doctor may receive a notification 308 and a further encryption step 310 to decrypt data 310. In operation, the doctor opens the app using 216 and continues to chat with patient at module 312.

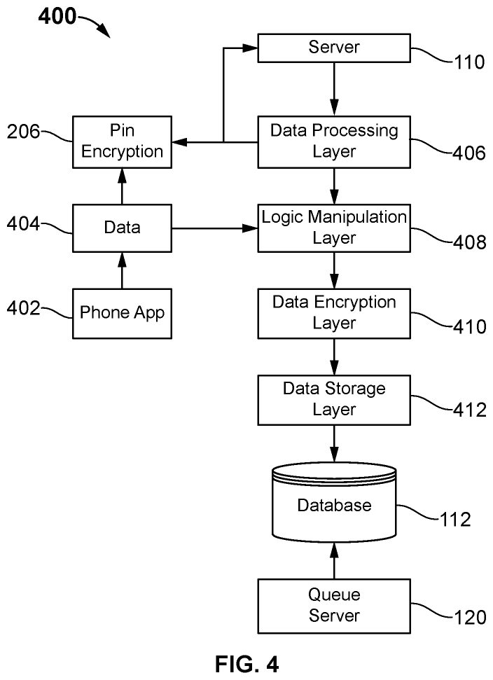

With reference now to FIG. 4, an exemplary schematic diagram for registration and security of the system for scheduling and managing healthcare and telehealth services is shown generally at 400. In operation, a user may open the application 402 and input user data 404. PIN encryption module 206 is provided (utilizing the encryption server 114) is configured to communicate with multiple databases and encrypt patient information in a HIPPA compliant manner. The server 110 is configured with a data-processing layer 406, logic manipulation layer 408, data encryption layer 410, data storage layer 412, and database 112. Each is in communication with the queue server 120. In one exemplary embodiment, the data processing layer 406 and logic manipulation layer 408 are configured to s a handle massive quantities of data by taking advantage of both batch and stream-processing methods. This balances latency, throughput, and fault-tolerance by using batch processing to provide comprehensive and accurate views of batch data, while simultaneously using real-time stream processing to provide views of online data. Data encryption layer 410, executed by the processor, encrypts and/or decrypts the data for storage layer 412 and database 112 during use of the application.

In operation, the user and physician can register by inputting credentials including, but not limited to, first name, last name, email address, phone number, medical record number, healthcare organizations, appointment date, appointment time, password, and touch or face ID log-in. A physician enable registration for its users/patients automatically to avoid patients having to input the information themselves, as some patients are not able to do so or use mobile applications. The registration module allows the user to provide communication protocol with the user's health insurances provider (HMO or PPO) in a HIPPA compliant manner and provides patient background such as allergies and other requirements. The user to book an appointment with the features including, but not limited to, calendar for date navigation, appointment addition option, list of unavailable appointments indicated by a color, and appointment cancellation and rescheduling options. The application may notify the doctor or patient in case of change in appointments, cancellation of appointments, revised date and time from any end of user such as a patient. In one embodiment, the notification could be sent by means, including, but not limited to, SMS, email, or push message. The system further allows the user to input current conditions, and may comprise a telehealth sub-module component for communication with a physician or clinician which is stored in the database 112.

Referring now to FIG. 5 an exemplary back-end system for scheduling and managing healthcare and telehealth services is provided at reference numeral 500. Patient detail module 502 may comprise inputs of patient ID, user ID blood group, marital status, height, weight, emergency contact, case manager, insurance, and tribal status. User detail module 504 comprises non-medical information such as user ID gender email login date of birth social security and the like. Doctor (or physician) module 506 comprises inputs such as identification, user ID, certificate number, specialty, education, peer appointment charge. All of the information is stored in a plurality of databases.

Patient appointment module 508 combines information from modules 502, 504 and 506 to book appointments or telehealth visits from a physician. Data includes patient ID, purpose of visit, appointment date, patient name, mobile device IP address and the like. A prescription module is further provided 512 and comprises a doctor ID, medicine name, medicine routine and any additional notes such as treatment days and pills per day. This allows the doctors to write prescriptions using the system in an automated fashion. Appointment scheduling module 514 comprises utilizing patient ID and doctor ID and is configured to match patients and physicians based on the variables discussed in relation to FIG. 1 and FIG. 2. User chat module 518 is provided for chat features described with relation to FIG. 3 and FIG. 4.

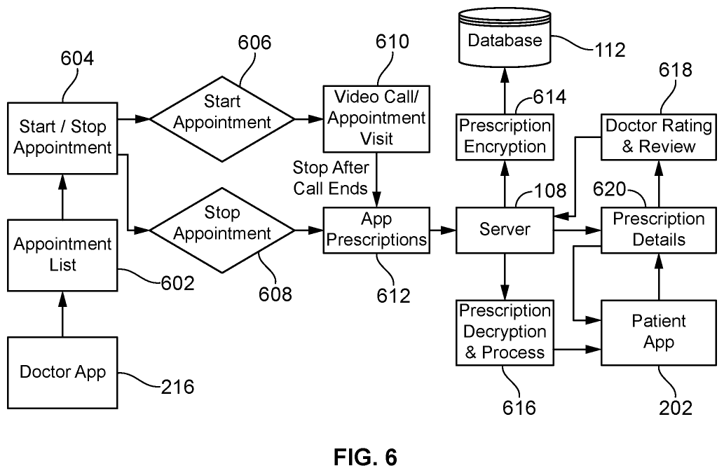

Referring now to FIG. 6, a step-wise method diagram for scheduling and managing healthcare and telehealth services according to an embodiment of the present invention shown at 600, more specifically, for booking an appointment or a telehealth conference. In operation, a doctor can open the application to view appointment list 602. Whether or not the doctor is seeing the patient in person or a telehealth conference, the doctor has the controls to start the appointment 610 or stop the appointment 608 at any time. If the doctor starts the appointment, the application will toggle to appointment,

And the queue server will ascertain whether the appointment should be booked as an individual appointment or a telehealth conference based on the variables. After the visit at module 610, the doctor can use the application to write a prescription 612 on the application for user by the user and a pharmacy if needed. In this way the doctor can input the prescription into the server which will run through encryption module 614 and a decryption process 616, all of which may be stored in the database 112. A doctor review and rating module 618 is provided in communication with prescription detail module 620 such that the doctor application has the ability to write and view prescriptions.

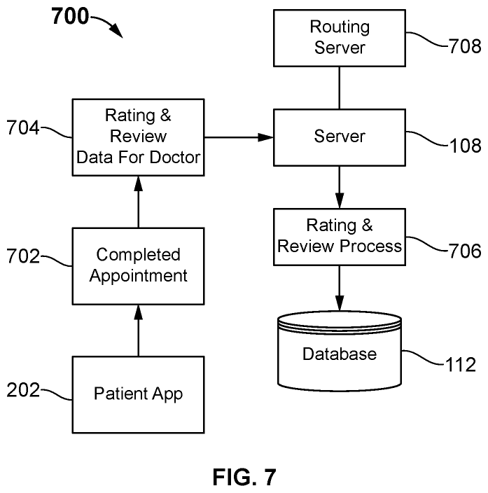

FIG. 7 shows systematic diagram of a variable server for rating doctors and storing ratings and routing the data to the database(s). In operation, the patient at 702 may be open and once the appointment is completed at 702, write rating and review module populates the UI to rate the doctor. The information is sent to server for the rating and review process 706 using routing server 708 uses database 112.

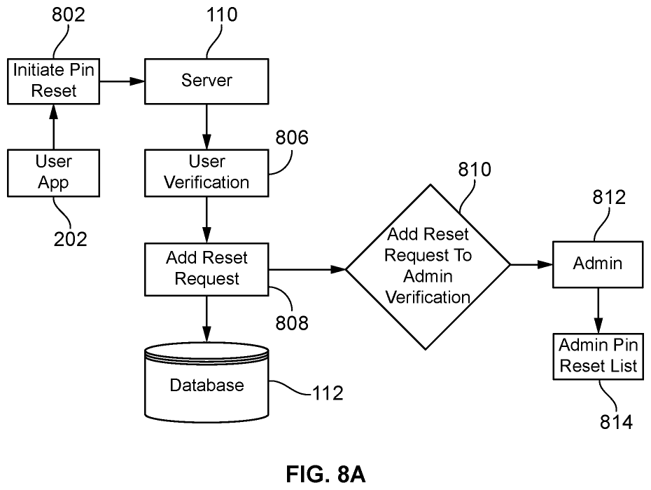

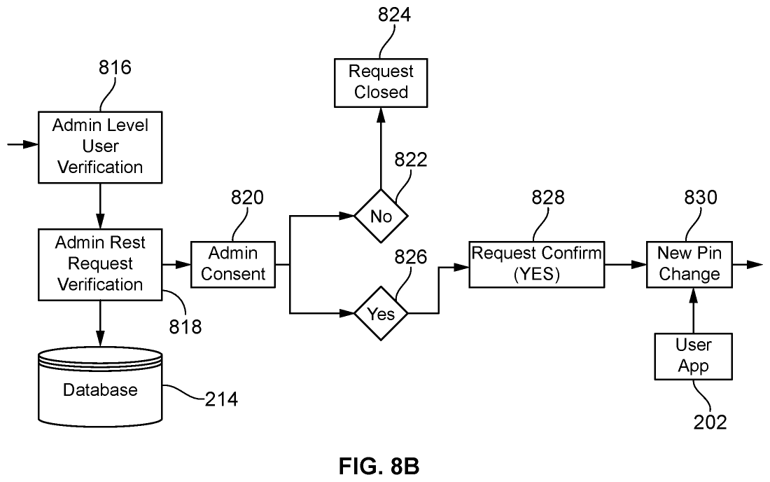

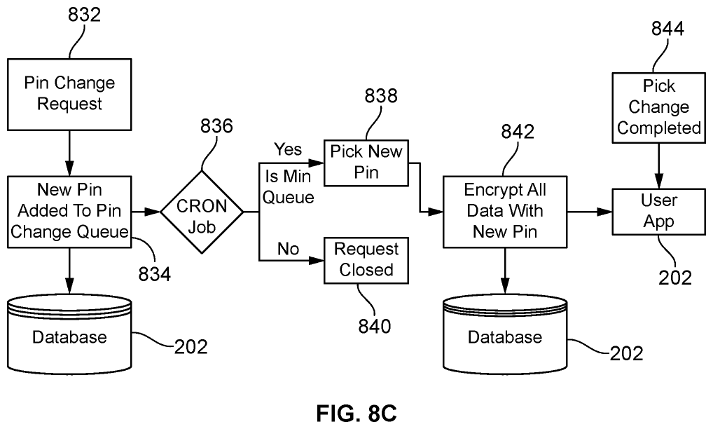

With reference now to FIG. 8a, a step-wise method diagram for scheduling and managing healthcare and telehealth services is shown generally at 800. At step 802, the user initiates or opens the application. Initiate PIN step 804 comprises encrypting input data and decrypting as needed. Further, once the application is opened the system may automatically finding a geographical location of the user and matching it to doctor profiles that are in area, or the last known location of the user based on previously found location data. Authentication step 806 comprises ensuring the user is validated or verified for the application step 807. At step 808, once the user is validated, the system outputs a request which is housed in database 112, and the request has been to administrator (admin) verified for authentication step 810. If the admin authenticates, the administrator 812 takes control may reset the PIN for encryption. Referring now to FIG. 8b, the system performs an administer level user verification step 816 and the administrator request reset verification step 818. At step 820, the admin consents to allow the user into the system step 820. If no consent is given at step 820, the request is closed at step 824. If consent is given at step 820 the system request confirmation from the user step 828 and a PIN is changed at step 830. The user application is then notified. Referring now to FIG. 8c, once the PIN change request is input at step 830, a new PIN is added to PIN change queue step 830. These are stored in database 202. The system is configured to generate a CRON job at step 836 (e.g., a process or task that runs periodically on a Unix system. Some examples of crons include syncing the time and date via the Internet every ten minutes, sending an e-mail notice once a week, or backing up certain directories every month). If “yes” at step 838 the system generates a new PIN. After the new pin generated the system encrypts all data with the new PIN 842 to the user is notified in the pic change is completed step 844. If “no” the request is closed at step 840.

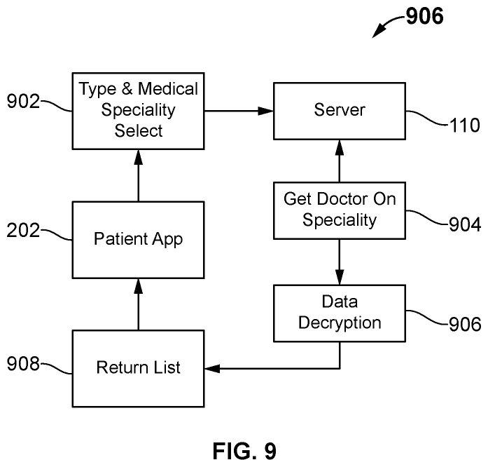

FIG. 9 shows another systematic diagram of a variable server according to an embodiment at 900. In operation, patient app 202 is opened and the user is able to select a type in medical specialty at module 902. This information is sent to server. A doctor specialty list is provided to the server on the doctor side and once decrypted returns the appropriate doctor 906.

Each smart device has user interface (UI) for registering a user on the system and inputting the credentials. Examples of credentials comprises first name, last name, email address, phone number, medical record number, healthcare organizations, appointment date, appointment time, password, and touch ID log-in. The UI allows the user to enter the email address, the password, and a first name respectively for accessing in the database through register option. In another embodiment of the present system, the user may further access in the database by using social media details such as Google® credentials or Facebook® through social media option. Preferred embodiments of this invention are described herein, including the best mode known to the inventors for carrying out the invention. It should be understood that the illustrated embodiments are exemplary only and should not be taken as limiting the scope of the invention.

Although specific features of various embodiments of the invention may be shown in some drawings and not in others, this is for convenience only. In accordance with the principles of the invention, the feature(s) of one drawing may be combined with any or all of the features in any of the other drawings. The words “including,” “comprising,” “having,” and “with” as used herein are to be interpreted broadly and comprehensively and are not limited to any physical interconnection. Moreover, any embodiments disclosed herein are not to be interpreted as the only possible embodiments. Rather, modifications and other embodiments are intended to be included within the scope of the appended claims.

I claim:

1. A system for integrating dynamic scheduling and management of appointments and telehealth conferences, the system comprising:
at least one client device associated with a user, wherein the at least one client device comprises a graphical user interface (GUI) that allows a user to input a request and a plurality of user variables;
a network for receiving the input request to the physician;
a management server in communication with the network, wherein the management server receives the input request from the at least one client device, and outputs a request to a provider-side server, wherein the provider-side server is communication with at least one physician device associated with a physician, and wherein the at least one physician device comprises a graphical user interface (GUI) that allows a physician to receive the request at a the physician device;
an encryption server in communication with the management server, wherein the encryption server is configured to encrypt and decrypt the user inputs; and
a queue server in communication with the management server and encryption server, wherein the queue server is configured to receive the plurality of variables, wherein the queue server comprises a global positioning (GPS) module configured to ascertain a location of the user and the physician, and wherein using the location and at least one of the plurality of variables, the queue server is configured to automatically determine whether the appointment is an in-person appointment or a telehealth appointment and notify both the user and the physician.

2. The system of claim 1, further comprising a database in communication with the queue server, wherein the database is configured to save and organize the plurality of variables input by the user and the physician.

3. They system of claim 1, wherein the variables comprise current user location, user last known location, past conditions, current conditions, history, characteristics of the user, physician specialties, ratings, location, last known location, or any combination thereof.

4. The system of claim 1, wherein the encryption server comprises a transport layer security (TLS), configured to provide communications security over a computer network in a HIPPA compliant.

5. The system of claim 2, wherein the GPS module is configured to communicate with the user device and the physician device to alert the management server that the user is out of network or out of cellular service, and further configured alert the server when the user is back in network or back in cellular service.

6. The system of claim 5, wherein the queue server is configured store messages in the database, and the management server is configured to poll the database for the user or the physician location information to ascertain a location of where the user or the physician may be based on the previous known locations, appointment locations, home addresses and any combination thereof.

7. The system of claim 1, wherein the encryption server comprises a first encryption module configured to load PIN encryption keys into each PIN before it transmits data.

8. The system of claim 7, wherein the encryption server further comprises a second encryption module configured to further encrypt the appointment data for an in person visit or telehealth conference.

9. The system of claim 7, wherein the encryption server further comprises a physician encryption module configured to further encrypt the physician side information.

10. The system of claim 1, wherein the management server is configured to automatically populates the user UI with a physician list with a chat module configured to allow the patient to chat with the physician via a process chat data encryption module.

11. A non-transitory computer-readable medium for storing instructions that, in integrating dynamic scheduling and management of appointments and telehealth conferences, when executed on one or more processors, cause the one or more processors to:
receive an input from at least one client device associated with a user, wherein the at least one client device comprises a graphical user interface (GUI) that allows the user to input a request and a plurality of user variables;
output, via a network a request to a provider-side server, wherein the provider-side server is communication with at least one physician device associated with a physician, wherein the at least one physician device comprises a graphical user interface (GUI) that allows a physician to receive the request at a physician device;
encrypt, via an encryption server in communication with the management server, the user inputs;
receive the plurality of variables at a queue server in communication with the management server and encryption server;
ascertain a location of a user and a physician;
use the location and at least one of the plurality of variables, automatically determine whether the appointment is an in-person appointment or a telehealth appointment and notify both the user and the physician.

12. The non-transitory medium of claim 11, when executed on one or more processors, cause the one or more processors to: save and organize the plurality of variables input by the user and the physician via communication with a database.

13. The non-transitory medium of claim 11, wherein the variables comprise current user location, user last known location, past conditions, current conditions, history, characteristics of the user, physician specialties, ratings, location and last known location.

14. The non-transitory medium of claim 11, wherein the encryption server comprises a transport layer security (TLS), configured to provide communications security over the computer network in a HIPPA compliant.

15. The non-transitory medium of claim 11, when executed on one or more processors, cause the one or more processors to communicate with the user device and the physician device to alert the management server that the user is out of network or out of cellular service, and further to alert the server when the user is back in network or back in cellular services.

16. The non-transitory medium of claim 11, when executed on one or more processors, cause the one or more processors to store messages in the database, poll the database for the user or the physician location information to ascertain a location of where the user or the physician may be based on the previous known locations, appointment locations and home address.

17. The non-transitory medium of claim 11, when executed on one or more processors, cause the one or more processors to encrypt the data using a first encryption module configured to load PIN encryption keys into each PIN before it transmits data.

18. The non-transitory medium of claim 11, when executed on one or more processors, cause the one or more processors to further encrypt the appointment data whether it be an in person visit or telehealth conference using a second encryption module.

19. The non-transitory medium of claim 11, when executed on one or more processors, cause the one or more processors to further encrypt the physician side information using a physician encryption module.

20. A method for integrating dynamic scheduling and management of appointments and telehealth conferences, incorporated in a system including a client device, and a server in communication with the client device, wherein the server comprises a memory to store instructions and a processor coupled with the memory to process the stored instructions, the method comprising the steps of:
receiving an input from at least one client device associated with a user, wherein the at least one client device comprises a graphical user interface (GUI) that allows the user to input a request and a plurality of user variables;
outputting, via a network a request to a provider-side server, wherein the provider-side server is communication with at least one physician device associated with a physician, wherein the at least one physician device comprises a graphical user interface (GUI) that allows a physician to receive the request at a physician device;
encrypting, via an encryption server in communication with the management server, the user inputs;
receiving the plurality of variables at a queue server in communication with the management server and encryption server;
ascertaining a location of a user and a physician;
using the location and at least one of the plurality of variables, automatically determine whether the appointment is an in-person appointment or a telehealth appointment and notify both the user and the physician.
